(19) United States
(12) Patent Application Publication
(10) Pub. No.: US 2008/0020064 A1
(43) Pub. Date: Jan. 24, 2008

(54) Methods and compositions for the treatment of infection or infectious colonization of the eyelid, ocular surface, skin or ear

(75) Inventor: Jeffrey P. Gilbard, Weston, MA (US)

Correspondence Address: PALMER & DODGE, LLP;KATHLEEN M. WILLIAMS, 111 HUNTINGTON AVENUE, BOSTON, MA, 02199, US

(73) Assignee: Advanced Vision Research

(21) Appl. No.: 11/490,917

(22) Filed: Jul. 21, 2006

(51) Int. Cl. A61K 33/14; A61K 31/522

(52) U.S. Cl. 424/661; 514/263.31

(57) ABSTRACT

The instant invention provides preparations comprising an oxidizing antimicrobial agent such as chlorine dioxide and a heterocyclic compound that improves the antibacterial effect of the oxidizing antimicrobial agent preparation. The invention has particular use as an eye care preparation such as an eye drop. The invention further provides methods for reducing bacterial colonization and treating infection.

### BACKGROUND OF THE INVENTION

A condition known as dry eye causes chronic eye irritation resulting from decreased tear production or increased evaporation that results in a loss of water from the tear film and an increase in tear film osmolarity. This increase in tear film osmolarity results in an osmotic dehydration of the surface associated with a decrease in the density of conjunctival goblet cells. Recently it has been shown that dry eye patients have increased bacterial colonization of their eyelids, and that the bacteria found in these patients decrease the proliferation of conjunctival goblet cells in tissue culture ((Graham et al Analysis of Bacterial Flora in Dry Eye, Ocular Surface, 3(1):S68, 2005). Dry-eye patients are also known to be more prone to eye infections, especially in the context of contact lens wear (Lemp MA “Is the dry eye contact lens wearer at risk?”, Cornea (United States), 1990, 9 Suppl 1 pS48-50; discussion S54).

Punctal plugs are a frequently used treatment for dry eye. They provide symptomatic relief for patients with dry eye, reduce elevated tear film osmolarity in the disease and reduce ocular surface staining. A problem with punctal plugs is that they are frequently colonized by pathogenic noncomensals, including Pseudomonas aeruginosa and Staphylococcus aureus, that may cause symptoms and increase the risk of eye infections (Soukiasian S H Microbiology of Explanted Punctal Plugs, ARVO Annual Meeting, Program#/Poster# 4981/B305, Apr. 29, 2004; Sugita J, Yokoi N, Fullwood N.J., et al. “The detection of bacteria and bacterial biofilms in punctal plug holes”, Cornea (United States), May 2001, 20(4) p362-5).

Another condition of clinical significance is inflammation of the eyelids and the eye surface. This inflammatory disorder, frequently resulting in symptoms of eye irritation, is called blepharitis. In a study involving 332 patients with blepharitis and 160 normal controls, it has been shown that blepharitis patients have greater quantities of bacteria on their eyelids compared to normal controls. This finding applied to patients with both anterior and posterior blepharitis (Groden L R, Murphy B, Rodnite J, et al. “Lid flora in blepharitis”, Cornea (United States), January 1991, 10(1) p50-3). Bacterial overgrowth has been hypothesized to contribute to the symptoms of blepharitis by the production of bacterial lipases and esterases that hydrolyze the wax and sterol esters in meibum, creating free fatty acids that are irritating to ocular tissue and may effect tear film stability (Ta CN, Shine W E, McCulley J P, et al. ‘Effects of minocycline on the ocular flora of patients with acne rosacea or seborrheic blepharitis’, Cornea (United States), August 2003, 22(6) p545-8). In addition, these fatty acids may promote eyelid and ocular surface inflammation (Shine W E, McCulley J P, Pandya A G ‘Minocycline effect on meibomian gland lipids in meibomianitis patients’, Exp Eye Res (England), April 2003, 76(4) p417-20).

Eye hygiene, including eye lid cleaning, has been recommended for all of these conditions or circumstances by eye doctors. Many of the treatments available for dry eye conditions are formulated as drops or ointments. However, drops and ointments have a limited lifespan once opened as they commonly become infected with microbial growth.

Chlorine dioxide has been called the ideal biocide. Despite the many advantages of chlorine dioxide, commercial use is limited because it is an unstable, highly reactive gas which is soluble in and decomposes in water. See, e.g., U.S. Pat. No. 4,941,917. Therefore, it has heretofore been necessary to generate aqueous chlorine dioxide solutions on site for immediate use or use within a relatively short time (typically less than an hour). Due to these limitations, chlorine dioxide has not met its full potential as an antimicrobial agent. Similarly, other oxidizing antimicrobial agents such as sodium perborate have been under utilized because of stability and effectiveness issues.

Therefore, a need exists for a preparation that can be used, for example, in or around the eye and is stable for extended periods of time.

### SUMMARY OF THE INVENTION

The instant invention is based on the discovery that eye care preparations comprising an oxidizing antimicrobial agent such as chlorine dioxide and a heterocyclic compound, i.e., dyphylline, exhibit beneficial effects. For example, preparations comprising chlorine dioxide and dyphylline are useful for the treatment and prevention of microbial infection and preservation of ocular products. The addition of chlorine dioxide and a heterocyclic compound preserve the solution for extended periods of time. Moreover, these preparations resist microbial growth prior to and after being used by an individual.

Accordingly, in at least one aspect, the instant invention provides eye care preparations comprising chlorine dioxide and a heterocyclic compound that improves the antibacterial effect of the chlorine dioxide preparation. The heterocyclic compounds can be bicyclic compounds such a xanthine. Exemplary heterocyclic compounds, include bicyclic compounds such as caffeine, theophylline, dyphylline, theobromine, xanthine, xanthinol, methylxanthine, and aminophylline. In a preferred embodiment, the bicyclic compound is dyphylline.

The chlorine dioxide in the preparations can be present in a concentration of about 25-125 ppm, about 50-75 ppm, or about 60 ppm.

The heterocyclic compound such as dyphylline can be present in 0.10-10%, 0.25-5% or about 1% to about 3%.

The preparations of the invention include solutions, creams, pastes, ointments, and gels. The preparations can be, for example, topical preparations that are applied to the eye, eye-lid, or eye margin. Further, the preparations are effective against bacteria, fungi, and/or yeast. The preparations of the invention may further comprise a pharmaceutically acceptable carrier.

In one exemplary embodiment, the preparation is a solution. The solutions of the invention can have an osmolality of 180 mOsm/Kg or less, or 165 mOsm/Kg or less.

An exemplary preparation of the invention is an eye care preparation having an osmolality of 165 mOsm/Kg or less and a chlorine dioxide concentration of about 50-75 ppm.

Another exemplary preparation of the invention comprises a solution comprising chlorine dioxide and dyphylline, wherein the chlorine dioxide is present at about 55-75 ppm and the preparation has a total osmolality of 165 mOsm/Kg or less.

Yet another exemplary preparation of the invention comprises, an eye drop comprising chlorine dioxide and dyphylline, wherein the chlorine dioxide is present at about 55-75 ppm and the preparation has a total osmolality of 165 mOsm/Kg or less.

The instant invention also provides methods of using the described preparations. For example, the invention provides a method of preventing or treating microbial infection in a subject by applying the preparations described herein to the infected area in an amount sufficient to prevent or treat a microbial infection in a subject. Exemplary microbial infections include bacterial, fungal and yeast infections.

The invention also provides methods of preventing or treating an infection of the ocular surface in a subject by applying the preparation to an ocular surface in an amount sufficient to prevent or treat an infection of the ocular surface in the subject. An exemplary infection of the ocular surface includes bacterial conjunctivitis. The invention also provides a method for reducing the risk of infection in patients, including dry-eye patients, wearing contact lenses.

The invention also provides methods of reducing the risk of infection of the eye in an eye surgery patient by applying the preparations described herein to an eyelid or ocular surface prior to a surgical procedure in an amount sufficient to reduce the risk of infection in the eye of a surgical patient. In accordance with this method, the preparation is applied multiple times over a number of days preceding the surgery.

The invention also provides methods of reducing the risk of infection of the eye in a subject wearing a punctal plug by applying the preparation described herein to the eye surface, the eyelid margin, the eyelid or the punctal plugs or into the tear film in an amount sufficient to reduce the risk of infection in the eye of the subject wearing a punctal plug.

The invention also provides methods for treating or reducing the risk of infection in a subject by applying the topical preparation described herein to the area that is infected or at risk of becoming infected in an amount sufficient to treat or reduce the risk of infection.

The invention also provides methods of reducing colonization or of treating an infection of the mucosal tissue or the epithelial tissue in a subject by applying the preparation described herein to the mucosal tissue or the epithelial tissue in an amount sufficient to treat the infection in the subject.

The invention also provides a method of treating dry eye in a subject by applying the eye care preparation described herein to the eye or eyelid in an amount sufficient to treat dry eye in the subject.

The invention also provides a method of increasing goblet cell density and mucosal epithelial membrane mucin expression in a subject by applying the eye care preparation described herein to the eye in an amount sufficient to increase goblet cell density and mucosal epithelial membrane mucin expression. This increase can be determined using rose Bengal or other vital staining techniques. In dry eye, the ocular surface stains less with an increase in conjunctival goblet cell density and mucosal epithelial membrane mucin expression.

The invention also provides methods for sterilizing a surface by contacting the surface with the preparation described herein, or incorporating within a surface the preparation described herein, in an amount sufficient to sterilize the surface. Exemplary surfaces include surfaces on a body, a medical instrument, or a medical device. An exemplary medical device includes a contact lens.

The invention also provides kits for the treatment or prevention of a microbial infection, comprising the preparation described herein and instruction for use. The invention also provides kits for the treatment of an ocular disorder comprising the antimicrobial preparation described herein and instruction for use. The kits may further comprise an applicator.

### DETAILED DESCRIPTION OF THE INVENTION

At present, there exists a need for compositions and methods for preventing microbial growth and treating microbial infections. In particular, there exists a need for compositions for preventing microbial growth in products for use in the eye or surrounding area. In particular, the need exists for compositions that remain free of microbial growth for extended periods of time, i.e., compositions that contain a preservative that inhibit microbial growth and that do not irritate the area to which it is applied. Preferably, the compositions will contain an antimicrobial composition that will kill or retard the growth of microbes (e.g., a bactericidal or bacteriostatic composition). In certain embodiments, the compositions are also useful for treating or preventing infection of, for example, the skin. In further embodiments, the preparations of the invention are useful in treating or preventing infection on surfaces, medical devices, and medical instruments.

### DEFINITIONS

The invention will be described with reference to the following definitions that, for convenience, are collected here.

The term “cleaning an eyelid” is used herein to describe the act of significantly reducing the amount of dirt, debris, or bacteria, from an eyelid.

The term “dry eye” is known in the art as a condition of a subject that has a loss of water from the tear film due to either a decrease in tear production or an increase in tear film evaporation. Tear production can decrease from lacrimal gland disease, including, but not limited to, that which occurs in Sjögren's syndrome, or from anything that decreases corneal sensation. Examples of conditions that decrease corneal sensation include, but are not limited to, diabetes, long-term contact lens wear, and corneal surgery, including LASIK eye surgery. Tear film evaporation can increase from meibomian gland dysfunction, that manifests itself by stenosis or closure of the meibomian gland orifices, or in the presence of large palpebral fissure widths. Causes for large palpebral fissure width include, but are not limited to, normal biological variation and thyroid eye disease. Dry eye is often an age related disease, and may also be caused by a dietary deficiency of omega-3 essential fatty acids. Dry eye is associated with bacterial overcolonization of the eyelids.

The term “eyelid” as used herein, includes the tarsal conjunctival surface, both the interior and exterior surfaces of the eyelid, the eyelid margin, the glands in and around the eyelid margins, the hair follicles of the eyelid, the eyelashes, and the periocular skin surrounding the eye.

The term “eye surface inflammatory disorder,” as used herein, is intended to include disorders associated with eye surface inflammation. These disorders include dry eye, where ocular surface inflammation has been demonstrated, as well as both anterior and posterior blepharitis. In anterior blepharitis, the inflammation is centered around the eyelashes. Posterior blepharitis or meibomitis is associated with inflammation of the tarsal and bulbar conjunctiva, and complicated by hordeolums and chalazions, and leads to meibomian gland dysfunction. Both anterior and posterior blepharitis are associated with bacterial overcolonization of the eyelids.

Animal models with combined dry eye and eye surface inflammatory disorder have been produced, and can be used to test the efficacy of the antimicrobial preparations provided herein. For example, a rabbit model for meibomianitis and meibomian gland dysfunction has been developed. In this animal model, meibomian gland orifice closure results in the development of inflammation around the meibomian glands (i.e., meibomianitis), inflammation in the eyelids (blepharitis), inflammation in the conjunctiva (conjunctivitis) and in an increase in tear film osmolarity and a decrease in the levels of corneal glycogen and conjunctival mucus-containing goblet cells characteristic of dry-eye surface disease.

The term “eyelid disorder” is defined as a disorder that results in inflammation of the eyelashes and/or eyelash follicles and/or eyelid margins, or inflammation of the lipid producing glands that are located in the eyelid, including meibomianitis and anterior blepharitis. Exemplary eyelid disorders include, but are not limited those caused by bacterial infection.

The term “ocular disorder” as used herein, includes ocular surface disorders, disorders of the eyeball, periocular skin disorders, and eyelid disorders. Exemplary ocular disorders include, but are not limited to, dysfunctions of the tear film, inflammation of the eyelid margins due to bacterial infection, infections inside the eye known as endophthalmitis, and dry eye.

The term “treatment” as used herein is defined as prophylactic treatment (e.g., daily preventative use) or therapeutic treatment (e.g., a single treatment or a course of treatment) of a subject with or at risk for an ocular disorder, or with an ear or skin condition, that are associated with or exacerbated by infections or bacterial colonization.

The term “preparation or antimicrobial preparation” as used herein includes compositions comprising an oxidizing antimicrobial compound, for example, chlorine dioxide, and a heterocyclic compound that stabilizes the preparation. The preparation of the invention can be a solution, cream, paste, ointment, gel or the like. The preparations of the invention can be applied to, for example, the skin, eye, or eyelid.

The term “heterocyclic compound that improves the antibacterial effect of the chlorine dioxide preparation” is intended to include heterocyclic compounds that, when combined with chlorine dioxide, provide beneficial effects. For example, the heterocyclic compound may increase the half-life of the chlorine dioxide in the preparation, may provide beneficial treatment effects, may increase the efficacy of the preparation, etc. In an exemplary embodiment, the heterocyclic compound improves the bactericidal effect of chlorine dioxide in the compositions, thereby extending the time that the composition is free of microbial growth. Exemplary heterocyclic compounds are xanthenes. Specific exemplary heterocyclic compounds include caffeine, theophylline, dyphylline, theobromine, xanthine, xanthinol, methylxanthine, and aminophylline. In a preferred embodiment, the heterocyclic compound used in the preparations of the invention is dyphylline.

As used herein the language “pharmaceutically acceptable carrier” is intended to include any and all solvents, agents, and the like, compatible with pharmaceutical administration. The use of such media and agents for pharmaceutically active substances is well known in the art. Except insofar as any conventional media or agent is incompatible with the antimicrobial preparations described herein, such media can be used in the compositions of the invention. Pharmaceutical compositions suitable for topical application preferably take the form of a drop, solution, ointment, cream, lotion, paste, gel, spray, aerosol, or oil. Exemplary carriers which may be used include water, carboxymethylcellulose, petroleum jelly, mineral oil, lanolin, polyethylene glycols, alcohols, and combinations of two or more thereof.

### Methods and Compositions

Effective health and cleanliness of an eye is dependant upon the ability to control the level microbes. Accordingly, compositions that have extended utility once opened, i.e., that resist microbial growth, are useful for the treatment of the eye.

The present invention provides compositions and methods, which decrease, e.g., significantly decrease, the number of microbes present in or around, for example, an eye, or in materials used in or around the eye.

Accordingly, the invention is directed to a preparation comprising an antibacterial concentration of chlorine dioxide and a heterocyclic compound that improves the antibacterial effect of the chlorine dioxide preparation. The preparation may also contain a pharmaceutically acceptable carrier or water. The preparation may be used as a preservative for materials used in conjunction with the eye such as eye drops or may be specifically formulated for the treatment of a particular disorder, e.g., an ocular disorder selected from blepharitis, dry eye, infectious conjunctivitis, an ear infection, or a skin infection. The preparation may also be used to sterilize, for example, surgical instruments, medical indwelling devices, surfaces and the like. The preparation may also be incorporated into the surface of medical devices for sustained release of the preparation. One of skill in the art would understand that the preparation of the invention may be prepared in the form of drops, solution, paste, cream, foam, gel, ointment, or the like, or incorporated into sustained-release carriers such as sustained-release polymers, liposomes and microcapsules.

There are several commercial generators for producing the chlorite/chlorine chlorine dioxide. Suitable generators are disclosed in U.S. Pat. Nos. 4,247,531; 5,204,081; 6,468,479; and 6,645,457, the disclosures of which are incorporated herein by reference.

Chlorine dioxide can be produced with high efficiency by reducing sodium chlorate in a strong acid solution with a suitable reducing agent (for example, hydrogen peroxide, sulfur dioxide, or hydrochloric acid):

2ClO₃⁻+2Cl⁻+4H⁺→2ClO₂+Cl₂+2H₂O

Alternatively, chlorine dioxide can be made by one of three methods using sodium chlorite: The sodium chlorite—chlorine gas method (2 NaClO₂+Cl₂→2ClO₂+2 NaCl); the sodium chlorite—hypochlorite method (2 NaClO₂+2 HCl+NaOCl→2 ClO₂+3 NaCl+H₂O); or the sodium chlorite—hydrochloric acid method (5 NaClO₂+4 HCl→5 NaCl+4 ClO₂). Finally, chlorine dioxide can be produced by electrolysis of a chlorite solution (NaClO₂+H₂O→ClO₂+NaOH+½ H₂). Preparations of the invention comprise between about 25-200 ppm of chlorine dioxide, about 50-150 ppm of chlorine dioxide, about 50-100 ppm of chlorine dioxide, or about 50-75 ppm of chlorine dioxide. For treatment of infection, higher values may be used.

The heterocyclic compound such as dyphylline can be present in 0.10-10%, 0.25-5% or about 1% to about 3%.

In certain embodiments, the antimicrobial preparation is an aqueous solution containing chlorine dioxide as described herein. The solutions of the invention can have an osmolality of, for example, about 180 mOsm/Kg, about 175 mOsm/Kg or less, about 170 mOsm/Kg, about 165 mOsm/Kg or less, about 160 mOsm/Kg or less, or about 155 mOsm/Kg or less.

An exemplary preparation of the invention is a preparation having an osmolality of 165 mOsm/Kg or less and a chlorine dioxide concentration of about 50-75 ppm.

The preparations may further include buffers, solubilizers, viscosity increasing agents, preservatives, anti-inflammatory agents and salts.

In a preferred embodiment, the invention provides an eye drop comprising chlorine dioxide and a heterocyclic compounds. The eye drop includes a balance of electrolytes found in natural tear fluid required for ocular surface maintenance, function and repair. These electrolytes are present in amounts and proportions sufficient to maintain or restore conjunctival goblet cells and corneal glycogen, thereby maintaining mucus-mediated lubrication and the potential for normal healing. This enables topical application of the preparation to ocular surfaces preferably without substantially reducing the density of conjunctival mucus-containing goblet cells or levels of corneal glycogen. Goblet cells form a critical layer of the tear film, providing the eye surface with lubrication, and playing an important role in the system that traps foreign matter that may enter the eye, and promptly removes it. Corneal glycogen is the energy source for the sliding step in corneal wound healing. Their preservation is therefore important in maintaining the health of ocular surfaces.

The eye drop compositions of the invention include, in addition to chlorine dioxide and a heterocyclic compounds, e.g., dyphylline, a balance of electrolytes naturally found in tear fluid. These electrolytes principally include major amounts of sodium and chloride, and lesser amounts of potassium and bicarbonate.

The preparation may also contain other naturally-occurring elements of the tear fluid, such as proteins, enzymes, lipids and metabolites as described in U.S. Pat. No. 4,911,933. Typically, in an isotonic preparation, the potassium is present at a concentration of about 22.0 to 43.0 mM/l, the bicarbonate is present at a concentration of about 29.0 to 50.0 mM/l, the sodium is present at a concentration of about 130.0 to 140.0 mM/l, and the chloride is present at a concentration of about 118.0 to 136.5 mM/l, or the electrolyte components can be diluted to create hypotonic formulations where the ratios between the electrolyte concentrations remain unchanged.

The eye drop compositions can further optionally include calcium, magnesium and phosphate. In such embodiments, in isotonic preparations, the calcium is preferably present at a concentration of about 0.5 to 2.0 mM/l, the magnesium is preferably present at a concentration of about 0.3 to 1. 1 mM/l, and the phosphate is preferably present at a concentration of about 0.8 to 2.2 mM/l. For hypotonic formulations, the electrolyte components can be diluted to create hypotonic formulations where the ratios between the electrolyte concentrations remain unchanged.

Accordingly, in a particular embodiment, the invention provides an ophthalmic solution set forth in Table 2 of the Examples.

The pH of the ophthalmic preparation generally ranges from about 7.0 to 8.0, as measured by, for example, a Fisher pH Accumet Model 600. However, this pH range need not be rigidly adhered to, and it may be desirable to alter pH outside of this range, for instance, to improve ophthalmic drug penetration through the ocular surface. In view of the teachings provided herein, those skilled in the art may employ other pH ranges.

The eye care compositions of the invention can be applied to the ocular surface by various methods known in the art. For example, the preparation can be applied topically to the ocular surface as eye drops or ointments. The preparation can also be applied using an eye cup so that the eye is bathed. The preparation can also be applied using a continuous or near continuous infusion device for ocular surface irrigation and/or wetting and/or drug delivery. The preparation can also be applied by release from a sustained-release carrier such as a sustained-release polymer, a liposome or a microcapsule. The preparation may also be applied by devices that spray solutions as required onto the surface of the eye.

The invention is further directed to methods of using the compositions described above to treat a subject, e.g., a subject having or at risk of having an infection, e.g., an infection of the eye or skin. The method comprises the step of applying the antimicrobial preparation described herein to the site of the infection, or site where an infection is likely to occur, or the site from which an infection might originate, for a time and under conditions effective for reducing the amount of bacteria present. In a specific embodiment, the time and conditions selected result in an at least about 1 log reduction in colony-forming units of the infecting bacteria after one minute of exposure to the antimicrobial preparation. In other embodiments, the application of the antimicrobial preparation for one minute results in an at least about 2, 3, 4 or 5 log reduction in colony-forming units.

The invention also provides methods of treating ocular disorders such as blepharitis, dry eye, eye inflammatory disorders, infectious conjunctivitis, and other ocular disorders that result from or are complicated by bacterial colonization or infection of the eye or surrounding tissue, by applying the preparations provided herein to the eye and/or surrounding tissue of a subject.

The invention also provides methods of treating infection of the ocular surface by applying the antimicrobial preparations provided herein to the eye of a subject. Exemplary infections that can be treated with the antimicrobial preparations provided herein include conjunctivitis, e.g., infectious conjunctivitis and corneal ulcers.

The invention also provides methods of preventing an eye infection in a subject having an eye surgery or procedure. These methods would comprise applying the antimicrobial preparation to the eye over a number of days preceding the surgery or procedure to reduce or eliminate the risk of developing an infection during the surgery or procedure. Exemplary procedures include cataract or LASIK surgery.

The invention also provides methods of maintaining low bacterial colony counts on punctal plugs that have been placed in patients for treatment. Exemplary punctal plugs include those manufactured by Odyssey Medical (Memphis, Tenn.), and Eagle Vision (Memphis, Tenn.).

Application of the antimicrobial preparations set forth herein can be by any one of a number of art recognized methods. For example, application can be by a applicator, such as a Qtip or pad, by drops from a dropper or bottle, or using a finger or fingers.

The antimicrobial preparations of the invention may be applied one or more times per day, and may be left in place as long as needed, depending on the intended indication. The number of days which a subject applies the antimicrobial preparation, and the duration of the application, will depend on the intent of treatment or on the location and severity infection, and efficacy of the preparations on a given infection. In certain embodiments, the antimicrobial preparation may be applied for a period of 30 seconds, 45 seconds, 1 minute, 2 minutes, 3 minutes, 4 minutes, 5 minutes, or longer. The antimicrobial preparation can be applied by release from a sustained release carrier such as a sustained-release polymer, a liposome or a microcapsule. The ordinary skilled physician would be able to effectively prescribe a treatment regimen that will be effective in treating or preventing an infection in an individual.

The methods described above may further include a rinsing step after a recommended period of exposure. This step preferably comprises a simple water rinse. The antimicrobial preparation may be rinsed from the area to which it was applied with ample water after application, e.g., with a hand, finger or any moist pad or cloth suitable for this purpose.

In further embodiments, the invention provides disinfecting a surface. Exemplary surfaces include those on medical instruments, in medical facilities, on medical devices, and those in a home, e.g., in a kitchen or bathroom.

### Commercial Applications

The methods and compositions of the invention find numerous commercial applications that could beneficially utilize compliance enhancing methods and compositions for antibacterial applications. Consequently, the invention includes a kit comprising the compositions of the invention, e.g., a kit for the treatment of an infection, e.g., an ocular infection, an ocular disorder, eyelid hygiene. The kits optionally include an applicator. The preparation can be in the form of drops, solution, paste, cream, foam, gel, or ointment, or the like, when included in the kits of the invention.

The kit may optionally be packaged with instructions for use. The kit may optionally contain a dispenser or applicator, e.g., a sponge, to apply the antimicrobial preparations of the invention to the infected area of a subject.

### EXAMPLES

A study was conducted to evaluate the stability of three different formulations of the eye care preparations comprising chlorine dioxide. The basic formulations were prepared with either 0.005% chlorine dioxide alone, or 0.005% chlorine dioxide and 2.5% dyphylline. The study incorporated an evaluation of each formulation with and without autoclave sterilization in the manufacturing procedure.

The analysis consisted of both USP Antimicrobial Effectiveness (preservative efficacy) testing and analytical testing over a three month time frame, at three storage conditions: 5° C., 25° C./60% relative humidity (RH), and 40° C./75% RH. Analytical testing for chlorine dioxide content, pH, and osmolality was performed every two weeks for three months. The Antimicrobial Effectiveness was evaluated on a monthly basis on formulations that met the acceptance criteria for a Category 1 article at the initial Antimicrobial Effectiveness testing.

The analytical testing of the dyphylline/chlorine dioxide samples showed a pH drift upward in both the autoclaved and non-autoclaved formulations.

The initial Antimicrobial Effectiveness testing indicated that both autoclaved and non-autoclaved dyphylline/chlorine dioxide samples met the USP criteria at initial formulation. Samples of the autoclaved dyphylline/chlorine dioxide formulation were then tested after four months (all conditions) and met USP criteria for Antimicrobial Effectiveness.

In the chlorine dioxide formulations without dyphylline, the chlorine dioxide concentration showed no clear trend. The chlorine dioxide only formulation samples did not meet the acceptance criteria for the Antimicrobial Effectiveness testing during the initial testing and this portion of the study was discontinued.

During the execution of the initial study, a confirmatory study was performed to confirm the passing Antimicrobial Effectiveness results for the autoclaved dyphylline/chlorine dioxide sample. In addition to this sample, an autoclaved chlorine dioxide formulation and a formula based on example 5, U.S. Pat. No. 6,024,954 (consistent with Refresh) were prepared. All three were tested using the USP Antimicrobial Effectiveness testing, with the dyphylline/chlorine dioxide and the formulation based on example 5, U.S. Pat. No. 6,024,954 (consistent with Refresh) passing through 28 days, and the chlorine dioxide alone sample again failing at day 7.

### INTRODUCTION

The following variations of the formulations were prepared for this stability evaluation:

TABLE I
Chlorine Dioxide Formulation

| Chemical | Percent |
|---|---|
| CMC | 0.25% |
| Sodium Chloride | 0.22218% |
| Potassium Chloride | 0.07315% |
| Magnesium Chloride, Hexahydrate | 0.005% |
| Sodium Phosphate, Monobasic, | 0.0056% |
| Monohydrate | |
| Calcium Chloride, Dihydrate | 0.00644% |
| Boric Acid | 0.12555% |
| Sodium Borate, Decahydrate | 0.00008% |
| Sodium Bicarbonate | 0.109887% |
| Chlorine Dioxide | 0.005% |

dyphylline/chlorine dioxide sample. In addition to this sample, an autoclaved chlorine dioxide formulation was prepared as well as a formula based on example 5, U.S. Pat. No. 6,024,954 (consistent with Refresh) were prepared (formulation in Table 3).

TABLE 3
Formulation based on example 5, U.S. Pat. No. 6,024,954
(consistent with Refresh)

| Chemical | Percent |
|---|---|
| CMC | 0.50% |
| Sodium Chloride | 0.62% |
| Potassium Chloride | 0.14% |
| Magnesium Chloride_{;} Hexahydrate | 0.006% |
| Calcium Chloride, Dihydrate | 0.02% |
| Boric Acid | 0.20% |
| Chlorine Dioxide | 0.005% |

Samples of each formula were submitted for USP Antimicrobial Effectiveness testing.

TABLE 2
Dyphylline/Chlorine Dioxide Formulation

| Chemical | Percent |
|---|---|
| CMC | 0.25% |
| Sodium Chloride | 0.11542% |
| Potassium Chloride | 0.038% |
| Magnesium Chloride, Hexahydrate | 0.0026% |
| Sodium Phosphate, Monobasic, | 0.00291% |
| Monohydrate | |
| Calcium Chloride, Dehydrate | 0.0035% |
| Boric Acid | 0.06522% |
| Sodium Borate, Decahydrate | 0.00004% |
| Sodium Bicarbonate | 0.05708% |
| Chlorine Dioxide | 0.005% |
| Dyphylline | 2.5% |

Each formula was prepared in duplicate, with one formula prepared without autoclaving, and the other prepared with autoclaving.

When each formulation and initial analytical testing was completed, samples were submitted to microbiology for the initial Antimicrobial Effectiveness Testing. The challenge organisms were S. aureus, P. aeruginosa, E. coli, C. albicans, and A. niger.

The remainder of the bulk solutions was filled into low density polyethylene (LDPE) bottles and placed into the appropriate stability chambers. The autoclaved dyphylline/chlorine dioxide formulation had an additional test performed at approximately four months to correlate with the last Antimicrobial Effectiveness testing.

During the execution of the initial study, a confirmatory study was performed to confirm the passing Antimicrobial Effectiveness results for the autoclaved

### Equipment

325=/−2.5° C. Incubator, VWR S/N 1000590
22.5±/−2.5° C. Incubator, Forma S/N 32889120
Spectrophotometer, Spectronic 2ODT, S/N
3DU9337006 Centrifuge, ID 34440967
pH Meter, ID 0157189
Osmometer, ID T09390

### Materials

1. Staphylococcus aurcus, ATCC 6538
2. Pseudomonas aeruginosa, ATCC 9027
3. Escherichia coil, ATCC 8739
4. Candida albicans, ATCC 10231
5. Aspergillus niger, ATCC 16404
6. Soybean Casein Digest Agar, (SODA)
7. Sabouraud Dextrose Agar, (SDA)
8. D/E Neutralizing Broth, (DEB)
9. 0.1N Sodium Thiosulfate Volumetric Solution
10. Potassium Iodide
11.Starch Indicator Solution
12. 2.5N Hydrochloric Acid

### Experiments

After compounding the six formulations, samples were submitted for the initial USP Antimicrobial Effectiveness testing per USP and SOP-00181. All test samples were challenged with approximately 1.0×10 to 1.0×10⁶ cfu/mT, of S. aureus ATCC 6538, P. aeruginosa ATCC 9027, E. coli ATCC 8739, C. albicans, ATCC 10231 and A. niger, ATCC 16404. The organisms were inoculated into a 50 mL centrifuge tube containing 10 mL of test sample at Time=O. One (1) mL was aliquoted from each centrifuge tube for the following 4 weeks. The log reduction was determined by the plate count method after 7 14, 21 and 28 days by diluting in DEB from 10″¹ to 10⁻⁴ for bacteria/yeast and 10⁻1 to 1⁻′ for mold. The plates were then poured with the appropriate media and incubated. Bacterial plates were poured with SCDA and incubated at 32.5±²0.5° C.. Yeast/mold plates were poured with SDA and incubated at 22.5±2.5° C.. The initial antimicrobial effectiveness data was reviewed before any further testing for antimicrobial effectiveness was conducted. Only formulas that had met the acceptance criteria at the initial testing were followed beyond the initial testing.

The acceptance criteria for USP Antimicrobial Effectiveness for a Category 1 item are:

Bacteria—7 day—Not less than 1.0 log reduction from initial count
14 day—Not less than 3.0 log reduction from initial count
21 day—No specification
28 day—No increase from 14 days count at 28 days

Yeast/Molds—7 and 14 days—No increase from initial count
21 days—No specification
28 days—No increase from initial count
(No increase is defined as NMT 0.5 log₁₀)

Samples of each formulation were pulled from all storage conditions every two weeks and analyzed for chlorine dioxide, pH, and osmolality.

### Results

The analytical testing of the dyphylline/chlorine dioxide samples showed a pH drift upward in both the autoclaved and non-autoclaved formulations, with the higher temperatures demonstrating a more dramatic pH change (see Table 4). There were no apparent changes in osmolarity in all samples at all storage conditions (Table 5). The chlorine dioxide concentration in the dyphylline/chlorine dioxide samples showed no clear trend (Table 6).

For the antimicrobial effectiveness testing, the initial formulations (both autoclaved and non-autoclaved), met the acceptance criteria. For the last (four month) time point, samples of the autoclaved formulation at all storage conditions were tested for antimicrobial effectiveness. The samples of the autoclaved formulation stored at 5° C., 25° C. % 60% RH, and 40° C./75% RH all met the acceptance criteria for USP Antimicrobial Effectiveness after four months (see Tables 7-11).

TABLE 4
pH Results
Dyphylline/Chlorine Dioxide Formulation

| | Storage | Initial | 2 week | 1 month | 1.5 month | 2 month | 2.5 month | 3 month | 4 month |
|---|---|---|---|---|---|---|---|---|---|
| BCL273- | 5° C. | 7.69 | 7.69 | 7.81 | 7.87 | 7.86 | 7.91 | 7.85 | |
| 128A | 25° C./60% | | 8.00 | 8.09 | 8.15 | 8.16 | 8.24 | 8.20 | |
| (Dyphylline/ | RH | | | | | | | | |
| Chlorine | 40° C./75% | | 8.20 | 8.28 | 8.34 | 8.39 | 8.45 | 8.36 | |
| Dioxide - not | RH | | | | | | | | |
| autoclaved) | | | | | | | | | |
| BCL273-128B | 5° C. | 7.95 | 8.13 | 8.11 | 8.13 | 8.11 | 8.17 | 8.07 | 8.19 |
| (Dyphylline/ | 25° C./60% | | 8.18 | 8.21 | 8.24 | 8.25 | 8.30 | 8.20 | 8.36 |
| Chlorine | RH | | | | | | | | |
| Dioxide - | 40° C./75% | | 8.27 | 8.32 | 8.42 | 8.39 | 8.44 | 8.36 | 8.49 |
| autoclaved) | RH | | | | | | | | |

TABLE 5
Osmolality Results (mOsm/1 g)
Dyphylline/Chlorine Dioxide Formulation

| | Storage | Initial | 2 week | 1 month | 1.5 month | 2 month | 2.5 month | 3 month | 4 month |
|---|---|---|---|---|---|---|---|---|---|
| BCL273- | 5° C. | 156 | 157 | 157 | 158 | 156 | 153 | 154 | |
| 128A | 25° C./60% | | 156 | 154 | 156 | 153 | 152 | 153 | |
| (Dyphylline/ | RH | | | | | | | | |
| Chlorine | 40° C./75% | | 154 | 155 | 154 | 151 | 151 | 151 | |
| Dioxide - not | RH | | | | | | | | |
| autoclaved) | | | | | | | | | |
| BCL273- | 5° C. | 157 | 159 | .160 | 161 | 157 | 157 | 159 | 159 |
| 128B | 25° C./60% | | 157 | 158 | 159 | 156 | 156 | 159 | 158 |
| (Dyphylline/ | RH | | | | | | | | |
| Chlorine | 40° C./75% | | 158 | 157 | 158 | 155 | 154 | 155 | 156 |
| Dioxide - | RH | | | | | | | | |
| autoclaved) | | | | | | | | | |

TABLE 6
Chlorine Dioxide Results (ppm) Dyphylline/Chlorine
Dioxide Formulation

| | Storage | Initial | 2 week | 1 month | 1.5 month | 2 month | 2.5 month | 3 month | 4 month |
|---|---|---|---|---|---|---|---|---|---|
| BCL273- | 5° C. | 59.14 | 62.52 | 62.52 | 59.14 | 60.83 | 59.14 | 59.14 | |
| 128A | 25° C./60% | | 60.83 | 62.52 | 58.29 | 61.67 | 64.21 | 60.83 | |
| (Dyphylline/ | RH | | | | | | | | |
| Chlorine | 40° C./75% | | 61.67 | 63.36 | 59.14 | 60.83 | 57.45 | 59.98 | |
| Dioxide - not | RH | | | | | | | | |
| autoclaved) | | | | | | | | | |
| BCL273- | 5° C. | 59.98 | 62.52 | 59.14 | 60.83 | 62.52 | 62.52 | 67.58 | 65.05 |
| 128B | 25° C./60% | | 62.52 | 64.21 | 60.83 | 60.83 | 60.83 | 66.74 | 62.52 |
| (Dyphylline/ | RH I | | | | | | | | |
| Chlorine | 40° C./75% | | 62.52 | 65.90 | 60.83 | 61.67 | 61.67 | 59.14 | 60.83 |
| Dioxide - | RH | | | | | | | | |
| autoclaved) | | | | | | | | | |

TABLE 7
Antimicrobial Effectiveness
Dyphylline/Chlorine Dioxide - not autoclaved,
BCL273-128A, T = initial

| Organism | Day | Log reduction | Pass/Fail |
|---|---|---|---|
| S. aureus | 7 | >4.94 | Pass |
| | 14 | >4.94 | Pass |
| | 21 | >4.94 | N/A |
| | 28 | >4.94 | Pass |
| P. aeruginosa | 7 | >5.07 | Pass |
| | 14 | >5.07 | Pass |
| | 21 | >5.07 | N/A |
| | 28 | >5.07 | Pass |
| E. coli | 7 | >4.96 | Pass |
| | 14 | >4.96 | Pass |
| | 21 | >4.96 | N/A |
| | 28 | >4.96 | Pass |
| C. albicans | 7 | 1.06 | Pass |
| | 14 | 1.04 | Pass |
| | 21 | 2.74 | N/A |
| | 28 | 3.58 | Pass |
| A. niger | 7 | 0.91 | Pass |
| | 14 | 1.00 | Pass |
| | 21 | 1.61 | N/A |
| | 28 | 1.05 | Pass |

TABLE 8
Antimicrobial Effectiveness
Dyphylline/Chlorine Dioxide - autoclaved. BCL273-128B, T = initial

| Organism | Day | Log reduction | Pass/Fail |
|---|---|---|---|
| S. aureus | 7 | >4.94 | Pass |
| | 14 | >4.94 | Pass |
| | 21 | >4.94 | N/A |
| | 28 | >4.94 | Pass |
| P. aeruginosa | 7 | 4.47 | Pass |
| | 14 | >5.07 | Pass |
| | 21 | >5.07 | N/A |
| | 28 | >5.07 | Pass |
| E. coli | 7 | >4.96 | Pass |
| | 14 | >4.96 | Pass |
| | 21 | >4.96 | N/A |
| | 28 | >4.96 | Pass |
| C. albicans | 7 | 0.34 | Pass |
| | 14 | 1.04 | Pass |
| | 21 | 1.23 | N/A |
| | 28 | 1.97 | Pass |
| A. niger | 7 | 0.85 | Pass |
| | 14 | 1.05 | Pass |
| | 21 | 1.71 | N/A |
| | 28 | 1.64 | Pass |

TABLE 9
Antimicrobial Effectiveness
Dyphylline/Chlorine Dioxide - autoclaved, BCL 273-128B.
40° C./75% RH Storage, T = 4 month

| Organism | Day | Log reduction | Pass/Fail |
|---|---|---|---|
| S. aureus | 7 | >5.07 | Pass |
| | 14 | >5.07 | Pass |
| | 21 | >5.07 | N/A |
| | 28 | >5.07 | Pass |
| P. aeruginosa | 7 | >5.06 | Pass |
| | 14 | >5.06 | Pass |
| | 21 | >5.06 | N/A |
| | 28 | >5.06 | Pass |
| E. coli | 7 | >5.09 | Pass |
| | 14 | >5.09 | Pass |
| | 21 | >5.09 | NIA |
| | 28 | >5.09 | Pass |
| C. albicans | 7 | 0.04 | Pass |
| | 14 | 1.80 | Pass |
| | 21 | 2.00 | N/A |
| | 28 | 2.29 | Pass |
| A. niger | 7 | 1.05 | Pass |
| | 14 | 1.08 | Pass |
| | 21 | 1.14 | N/A |
| | 28 | 1.35 | Pass |

TABLE 10
Antimicrobial Effectiveness
Dyphylline/Chlorine Dioxide - autoclaved. BCL 273-128B,
250° C./60% RH Storage, T = 4 month

| Organism | Day | Log reduction | Pass/Fail |
|---|---|---|---|
| S. aureus | 7 | >5.07 | Pass |
| | 14 | >5.07 | Pass |
| | 21 | >5.07 | N/A |
| | 28 | >5.07 | Pass |
| P. aeruginosa | 7 | 4.46 | Pass |
| | 14 | >5.06 | Pass |
| | 21 | >5.06 | N/A |
| | 28 | >5.06 | Pass |
| E. coli | 7 | >5.09 | Pass |
| | 14 | >5.09 | Pass |
| | 21 | >5.09 | N/A |
| | 28 | >5.09 | Pass |
| C. albicans | 7 | 0.07 | Pass |
| | 14 | 0.98 | Pass |
| | 21 | 1.03 | N/A |
| | 28 | 1.37 | Pass |
| A. niger | 7 | 0.94 | Pass |
| | 14 | 0.94 | Pass |
| | 21 | 1.12 | N/A |
| | 28 | 1.20 | Pass |

TABLE 11
Antimicrobial; Effectiveness
Dyphylline/Chlorine Dioxide - autoclaved. BCL 273-128B,
5° C. Storage, T = 4 month

| Organism | Day | Log reduction | Pass/Fail |
|---|---|---|---|
| S. aureus | 7 | >5.07 | Pass |
| | 14 | >5.07 | Pass |
| | 21 | >5.07 | N/A |
| | 28 | >5.07 | N/A |
| P. aeruginosa | 7 | 3.81 | Pass |
| | 14 | >5.06 | Pass |
| | 21 | >5.06 | N/A |
| | 28 | >5.06 | Pass |
| E. coli | 7 | >5.09 | Pass |
| | 14 | >5.09 | Pass |
| | 21 | >5.09 | N/A |
| | 28 | >5.09 | Pass |
| C. albicans | 7 | 0.32 | Pass |
| | 14 | 0.94 | Pass |
| | 21 | 0.95 | N/A |
| | 28 | 1.24 | Pass |
| A. niger | 7 | 0.97 | Pass |
| | 14 | 1.05 | Pass |
| | 21 | 1.08 | N/A |
| | 28 | 1.35 | Pass |

### Chlorine Dioxide, Autoclaved and Non-Autoclaved:

During the 3 months of testing, pH of both autoclaved and non-autoclaved increased in all conditions (see Table 12). There were no apparent changes in osmolality for all samples at all storage conditions (Table 13). The chlorine dioxide concentration showed no clear trend (see Table 14). These samples did not meet specification for USP Antimicrobial Effectiveness for the initial testing (Tables 15 and 16).

TABLE 12
pH Results
Chlorine Dioxide Formulation

| | Storage | Initial | 2 week | 1 month | 1.5 month | 2 month | 2.5 month | 3 month |
|---|---|---|---|---|---|---|---|---|
| BCL273-161C | 5° C. | 7.77 | 7.95 | 8.00 | 8.13 | 8.04 | 7.80 | 8.14 |
| (Chlorine Dioxide - | 25° C./60% RH | | 8.11 | 8.24 | 8.35 | 8.32 | 8.36 | 8.44 |
| not autoclaved) | 40° C./75% RH | | 8.35 | 8.49 | 8.59 | 8.56 | 8.57 | 8.68 |
| | 55° C. | | 8.55 | 8.68 | 8.78 | 8.73 | 8.69 | 8.84 |
| BCL273-161D | 5° C. | 8.54 | 8.51 | 8.49 | 8.54 | 8.43 | 8.37 | 8.49 |
| (Chlorine Dioxide - | 25° C./60% RH | | 8.53 | 8.54 | 8.58 | 8.50 | 8.45 | 8.50 |
| autoclaved) | 40° C./75% RH | | 8.58 | 8.64 | 8.70 | 8.64 | 8.60 | 8.73 |
| | 55° C. | | 8.66 | 8.74 | 8.80 | 8.75 | 8.73 | 8.86 |

TABLE 13
Osmolality, Results (mOsm/kg)
Chlorine Dioxide Formulation

| | | | | | 1.5 | | 2.5 | |
|---|---|---|---|---|---|---|---|---|
| | Storage | Initial | 2 week | 1 month | month | 2 month | month | 3 month |
| BCL273-161C | 5° C. | 141 | 142 | 140 | 138 | 141 | 138 | 136 |
| (Chlorine Dioxide - | 25° C./60% RH | | 141 | 136 | 136 | 138 | 136 | 134 |
| not autoclaved) | 40° C./75% RH | | 139 | 135 | 134 | 134 | 135 | 134 |
| | 55° C. | | 139 | 135 | 136 | 137 | 136 | 137 |
| BCL273-161D | 5° C. | 144 | 146 | 143 | 143 | 144 | 145 | 142 |
| (Chlorine Dioxide - | 25° C./60% RH | | 146 | 143 | 143 | 144 | 143 | 144 |
| autoclaved) | 40° C./75% RH | | 146 | 142 | 142 | 143 | 142 | 141 |
| | 55° C. | | 145 | 143 | 141 | 145 | 150 | 148 |

TABLE 14
Chlorine Dioxide Results (ppm)
Chlorine Dioxide Formulation

| | Storage | Initial | 2 week | 1 month | 1-5 month | 2 month | 2.5 month | 3 month |
|---|---|---|---|---|---|---|---|---|
| CL273-1610 | 5° C. | 59.14 | 59.14 | 60.53 | 59.14 | 59.14 | 60.83 | 60.83 |
| (Chlorine Dioxide - | 25° C./60% RH | | 58.29 | 59.98 | 59.14 | 69.27 | 62.52 | 59.99 |
| not autoclaved) | 40° C./75% RH | | 59.14 | 60.83 | 61.67 | 65.90 | 62.52 | 57.45 |
| | 55° C. | | 59.14 | 60.83 | 64.21 | 65.90 | 6252 | 59.29 |
| BCL273-161D | 5° C. | 62.52 | 67.58 | 65.05 | 65.90 | 62.52 | 69.27 | 62.52 |
| (Chlorine Dioxide - | 25° C./60% RH | | 59.98 | 63.36 | 63.36 | 71.81 | 62.52 | 65.05 |
| autoclaved) | 40° C./75% RH | | 60.83 | 64.21 | 65.90 | 60.83 | 60.83 | 62.52 |
| | 55° C. | | 60.83 | 62.52 | 67.58 | 64.21 | 62.52 | 64.21 |

TABLE 15
Antimicrobial Effectiveness
Chlorine Dioxide - not autoclaved, BCL273-161 C, T = initial

| Organism | Day | Log reduction | Pass/Fail |
|---|---|---|---|
| S. aureus | 7 | >4.97 | Pass |
| | 14 | >4.97 | Pass |
| | 21 | Discontinued testing | |
| | 28 | | |
| P. aeruginosa | 7 | 0.78 | Fail |
| | 14 | 1.46 | N/A |
| | 21 | Discontinued testing | |
| | 28 | | |
| E. coli | 7 | 2.07 | Pass |
| | 14 | 3.16 | Pass |
| | 21 | Discontinued testing | |
| | 28 | | |
| C. albicans | 7 | 0.99 | Pass |
| | 14 | 1.94 | Pass |
| | 21 | Discontinued testing | |
| | 28 | | |
| A. niger | 7 | 1.81 | Pass |
| | 14 | 2.26 | Pass |

| | |
|---|---|
| 21 | Discontinued testing |
| 28 | |

TABLE 16
Antimicrobial Effectiveness
Chlorine Dioxide - autoclaved, BCL273-161D, T = initial

| Organism | Day | Log reduction | Pass/Fail |
|---|---|---|---|
| S. aureus | 7 | >4.97 | Pass |
| | 14 | >4.97 | Pass |
| | 21 | Discontinued testing | |
| | 28 | | |
| P. aeruginosa | 7 | 0.88 | Fail |
| | 14 | 2.08 | N/A |
| | 21 | Discontinued testing | |
| | 28 | | |
| E. coli | 7 | 1.53 | Pass |
| | 14 | 2.76 | Fail |
| | 21 | Discontinued testing | |
| | 28 | | |
| C. albicans | 7 | 0.97 | Pass |
| | 14 | 2.09 | Pass |
| | 21 | Discontinued testing | |
| | 28 | | |
| A. niger | 7 | 1.85 | Pass |
| | 14 | 2.95 | Pass |

| | |
|---|---|
| 21 | Discontinued testing |
| 28 | |

During the execution of the initial study, a confirmatory study was performed to confirm the passing antimicrobial results for the autoclaved dyphylline/chlorine dioxide sample. In addition to this sample, an autoclaved chlorine dioxide formulation and a formulation based on example 5 of U.S. Pat. No. 6,024,954, consistent with the Refresh product marketed by Allergan were prepared for the confirmatory studies. All three were analyzed in the USP Antimicrobial Effectiveness test, and the earlier tests were confirmed with the dyphylline/chlorine dioxide and the formulation based on example 5, U.S. Pat. No. 6,024,954 (consistent with Refresh) passing through 28 days, and the chlorine dioxide alone sample failing at day 7 (see Tables 17-19).

TABLE 17
Antimicrobial Effectiveness
Repeated Dyphylline/Chlorine Dioxide - autoclaved,
BCL285-040B, T = initial

| Organism | Day | Log reduction | Pass/Fail |
|---|---|---|---|
| S. aureus | 7 | >4.99 | Pass |
| | 14 | >4.99 | Pass |
| | 21 | >4.99 | N/A |
| | 28 | >4.99 | Pass |
| P. aeruginosa | 7 | >5.14 | Pass |
| | 14 | >5.14 | Pass |
| | 21 | >5.14 | N/A |
| | 28 | >5.14 | Pass |
| E. coli | 7 | 4.77 | Pass |
| | 14 | >5.07 | Pass |
| | 21 | >5.07 | N/A |
| | 28 | 5.07 | Pass |
| C. albicans | 7 | 0.17 | Pass |
| | 14 | 0.76 | Pass |
| | 21 | 0.99 | N/A |
| | 28 | 1.87 | Pass |
| A. niger | 7 | 2.01 | Pass |
| | 14 | 1.35 | Pass |
| | 21 | 1.97 | N/A |
| | 28 | 2.28 | Pass |

TABLE 18
Antimicrobial Effectiveness
Repeated Chlorine Dioxide - autoclaved, BCL285-040A, T = initial

| Organism | Day | Log reduction | Pass/Fail |
|---|---|---|---|
| S. aureus | 7 | >4.99 | Pass |
| | 14 | >4.99 | Pass |
| | 21 | >4.99 | N/A |
| | 28 | >4.99 | Pass |
| P. aeruginosa | 7 | 0.92 | Fail |
| | 14 | 2.20 | N/A |
| | 21 | 1.35 | N/A |
| | 28 | −0.44 | N/A |
| E. coli | 7 | 1.71 | Pass |
| | 14 | 1.96 | Fail |
| | 21 | 2.63 | N/A |
| | 28 | 2.51 | N/A |
| C. albicans | 7 | 0.97 | Pass |
| | 14 | 1.72 | Pass |
| | 21 | 4.13 | N/A |
| | 28 | >5.28 | Pass |
| A. niger | 7 | 2.18 | Pass |
| | 14 | 2.89 | Pass |
| | 21 | 3.18 | N/A |
| | 28 | 3.51 | Pass |

TABLE 19
Antimicrobial Effectiveness
Formula based on example 5. U.S. Pat. No. 6,024,954
(consistent with Refresh)

| Organism | Day | Log reduction | Pass/Fail |
|---|---|---|---|
| S. aureus | 7 | >4.99 | Pass |
| | 14 | 4.99 | Pass |
| | 21 | >4.99 | N/A |
| | 28 | >4.99 | Pass |
| P. aeruginosa | 7 | >5.14 | Pass |
| | 14 | >5.14 | Pass |
| | 21 | >5.14 | N/A |
| | 28 | >5.14 | Pass |
| E. coli | 7 | >5.07 | Pass |
| | 14 | >5.07 | Pass |
| | 21 | >5.07 | N/A |
| | 28 | >5.07 | Pass |
| C. albicans | 7 | >5.28 | Pass |
| | 14 | >5.28 | Pass |
| | 21 | >5.28 | N/A |
| | 28 | >5.28 | Pass |
| A. niger | 7 | 1.18 | Pass |
| | 14 | 1.10 | Pass |
| | 21 | 1.00 | N/A |
| | 28 | 1.31 | Pass |

### CONCLUSION

Samples of the dyphylline/chlorine dioxide formulation stored at 5° C., 25° C./60% RH and 40° C./75% RH met the criteria for USP Antimicrobial Effectiveness for a Category 1 item after four months of storage.

The chlorine dioxide only formulation samples did not meet the acceptance criteria for the USP Antimicrobial Effectiveness testing for a Category 1 item.

The confirmatory Antimicrobial Effectiveness testing did confirm the observed results of the initial Antimicrobial Effectiveness testing for the dyphylline/chlorine dioxide and chlorine dioxide formulations. The dyphylline/chlorine dioxide formulation again met the acceptance criteria for USP Antimicrobial Effectiveness for a Category 1 item. The chlorine dioxide formulation did not meet the acceptance criteria for USP Antimicrobial Effectiveness for a Category 1 item. During the same testing, the formulation based on example 5, U.S. Pat. No. 6,024,954 (consistent with Refresh) also met the acceptance criteria for USP Antimicrobial Effectiveness for a Category 1 item.

### Incorporation by Reference

The contents of all references, patents, pending patent applications and published patents, cited throughout this application are hereby expressly incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A preparation comprising an oxidizing antibacterial agent and a heterocyclic compound that improves the antibacterial effect of the oxidizing agent.

2. The preparation of claim 1, wherein the heterocyclic compound is a bicyclic compound.

3. The preparation of claim 2, wherein the heterocyclic compound is a xanthine.

4. The preparation of claim 2, wherein the bicyclic compound is selected from the group consisting of caffeine, theophylline, dyphylline, theobromine, xanthine, xanthinol, methylxanthine, and aminophylline.

5. The preparation of claim 4, wherein the bicyclic compound is dyphylline.

6. The preparation of claim 1 wherein said oxidizing antibacterial agent is chlorine dioxide.

7. The preparation of claim 6, wherein the chlorine dioxide is present in a concentration of about 25-125 ppm.

8. The preparation of claim 7, wherein the chlorine dioxide is present in a concentration of about 50-75 ppm.

9. The preparation of claim 8, wherein the chlorine dioxide is present in a concentration of about 60 ppm.

10. The preparation of claim 1, wherein the preparation is in the form of a solution, cream, paste, ointment, or gel.

11. The preparation of claim 1, wherein the preparation is incorporated into a sustained-release carrier.

12. The preparation of claim 11 wherein said sustained-release carrier is selected from the group consisting of a sustained-release polymer, a liposome and a microcapsule.

13. The preparation of claim 1, wherein the preparation is a solution.

14. The preparation of claim 13, wherein the preparation has an osmolality of 180 mOsm/Kg or less.

15. The preparation of claim 14, wherein the preparation has an osmolality of 165 mOsm/Kg or less.

16. The preparation of claim 6, wherein the preparation has an osmolality of 165 mOsm/Kg or less and wherein the chlorine dioxide is present in a concentration of about 50-75 ppm.

17. The preparation of claim 1, wherein the preparation is for application to the eye, eye-lid, eye margin, punctal plug or a contact lens.

18. The preparation of claim 1, wherein the preparation is effective against at least one of the microbes selected from the group consisting of bacteria, fungi, and yeast.

19. The preparation of claim 1, wherein the preparation promotes an increase in conjunctival goblet-cell density in dry eye.

20. The preparation of claim 1, wherein the preparation promotes an increase in conjunctival goblet-cell density in eye surface inflammatory disorders.

21. The preparation of claim 1, further comprising a pharmaceutically acceptable carrier.

22. An antimicrobial preparation in the form of a solution comprising chlorine dioxide and dyphylline, wherein the chlorine dioxide is present at about 55-75 ppm and the antimicrobial preparation has a total osmolality of 165 mOsm/Kg or less.

23. A topical preparation comprising chlorine dioxide and dyphylline.

24. An eye care preparation comprising chlorine dioxide and dyphylline, wherein the chlorine dioxide is present at about 55-75 ppm

25. The eye care preparation of claim 24, wherein the eye care preparation is an eye drop in the form of a solution.

26. The eye care preparation of claim 24, wherein the eye care preparation in the form of a gel.

27. A preparation comprising a chlorite generating agent and dyphylline.

28. A method of treating a microbial colonization or infection in a subject comprising;
applying the preparation of claim 1 to the colonized or infected area in an amount sufficient to treat a microbial colonization or infection in a subject.

29. The method of claim 28, wherein the microbial colonization or infection is bacterial, fungal or yeast colonization or infection.

30. The method of claim 28, wherein the preparation is in the form of a drop, solution, cream, paste, ointment, or gel.

31. The method of claim 28, wherein the preparation is incorporated into a sustained-release carrier.

32. The method of claim 31 wherein said sustained-release carrier is selected from the group consisting of a sustained release polymer, a liposome and a microcapsule.

33. A method of treating a colonization or infection of the ocular surface or eyelid in a subject comprising;
applying the preparation of claim 1 to an ocular surface or eyelid in an amount sufficient to treat a colonization or infection of the ocular surface or eyelid in the subject.

34. The method of claim 33, wherein the infection is conjunctivitis.

35. The method of claim 34, wherein the conjunctivitis is infectious conjunctivitis.

36. The method of claim 33, wherein the colonization or infection is of a punctal plug.

37. The method of claim 33, wherein the colonization or infection is in conjunction with dry eye.

38. The method of claim 33, wherein the colonization or infection is in conjunction with an eye surface inflammatory disorder.

39. The method of claim 33, wherein the colonization or infection is in conjunction with an eyelid disorder.

40. The method of claim 33, wherein the ocular surface is in contact with a contact lens.

41. A method of reducing the risk of contact lens colonization or infection by applying the preparation of claim 1 to the surface of a contact lens prior to contact lens insertion in the eye.

42. A method of reducing the risk of infection of the eye in an eye surgery patient comprising:
applying the preparation of claim 1 to an eyelid or ocular surface prior to a surgical procedure in an amount sufficient to reduce the risk of infection in the eye of the surgical patient.

43. The method of claim 42, wherein the topical preparation is applied multiple times over a number of days preceding the surgery.

44. A method of treating colonization or an infection of the mucosal tissue or the epithelial tissue in a subject comprising:
applying the preparation of claim 1 to the mucosal tissue or the epithelial tissue in an amount sufficient to treat the colonization or infection in the subject.

45. A method of treating dry eye in a subject comprising:
applying the preparation of claim 1 to the eye or eyelid in an amount sufficient to treat dry eye in the subject.

46. A method of increasing goblet cell density and mucosal epithelial membrane mucin expression in a subject comprising:
applying the preparation of claim 1 to the eye or eyelid in an amount sufficient to increase goblet cell density and mucosal epithelial membrane mucin expression in the subject.
